(19) United States
(12) Patent Application Publication
(10) Pub. No.: US 2025/0014570 A1
(43) Pub. Date: Jan. 9, 2025

(54) Detection system and method for singing voice type determination

(71) Applicant: Shanxi University, Taiyuan City (CN)

(72) Inventors: Yang Yang, Taiyuan City (CN); Nanshu Feng, Taiyuan City (CN); Man Jin, Taiyuan City (CN)

(21) Appl. No.: 18/810,504

(22) Filed: Aug. 20, 2024

(30) Foreign Application Priority Data
Feb. 28, 2024 (CN) 2024102218495

(51) Int. Cl. G10L 15/02; G01H 9/00

(52) U.S. Cl. CPC G10L 15/02; G01H 9/00

(57) ABSTRACT

A detection system for singing voice type determination includes: a vibration measurement system, a data acquisition system and a data management system; wherein the vibration measurement system comprises: a laser scanning head, a morphology scanning unit, and an optical measurement system; the data acquisition system is a front-end with a built-in data acquisition and signal generator module; the data management system consists of a synchronized acquisition control module, a data acquisition module, an imaging display module, a data storage module, a data processing module, and an acoustic parameter extraction module. A method for singing voice type determination includes steps of: aligning and illuminating a laser to vocal cords; inputting an optical signal of vocal cord vibration into a data acquisition system, and then converting a frequency signal into velocity and displacement signals; and generating a real-time time-domain map and a spectrum map; then determining a peak frequency range of the vocal cord vibration.

### CROSS REFERENCE OF RELATED APPLICATION

The present invention claims priority under 35 U.S.C. 119 (a-d) to CN 202410221849.5, filed Feb. 28, 2024.

### BACKGROUND OF THE PRESENT INVENTION

### Field of Invention

The present invention relates to singing acoustics and acoustic measurements, and more particularly to a detection system and a method for singing voice type determination.

### Description of Related Arts

In the practice of vocal music teaching, teachers tend to use a comprehensive listening method (covering range, vocal quality, tone, etc.), sometimes supplemented with scientific identification means such as acoustic, photoelectric instruments and imaging technology, to determine the voice type of beginners. Even so, vocal judgment errors still occur frequently, resulting in students' laryngeal muscle tension, and even vocal cord nodules (mainly caused by improper or excessive use of the voice).

According to acoustic principle, if the vocal cords keep steady vibration during singing, the same vocal cords can imitate different tones of bel canto, nazionale cantanti and folk music when matched with different cavities. The determination of voice type is based on the peak frequency bandwidth of the vocal cords, while the diversity of vocal positions and cavities is precisely the interference in the determination of voice types.

According to vibration principle, the vocal cords, as the core acoustic components of the human singing system, form the vibrating body for the sound generation, or we can say, the sound source. When the vocal cords vibrate, the muscles around the vocal cords and the vocal cords themselves need to be relaxed and natural, and there should be no laryngeal tension, glottal insufficiency, or other problems. The acoustic property of vocal cords, like those of strings, has its own intrinsic frequency, including fundamental frequency and the harmonic frequency (an integer multiple of the fundamental frequency).

Conventionally, there are three methods to determine the singing voice type: the first one is based on the voice heard by teachers with auditory experience, which is actually the coupling result of vocal cord vibration and cavity resonance; the second one obtains the resonance peaks of singers by acoustic instrumentation, which is also based on the coupling of vocal cord vibration and cavity resonance; the third one adopts optoelectronic instrumentation and imaging technology, which can detect the morphological characteristics of the vocal cords (e.g., thickness, length, and color) and their vibrational properties (e.g., frequency, amplitude, phase, damping, etc.). However, because the subject needs to be fixed in a certain position during the operation in order to penetrate the instrument deep into his/her larynx, the singing state is unnatural and it is difficult to present the sound in the full range. In other words, the first two methods will be interfered by the cavity and other factors, and the voice heard by the teachers and the resonance peaks of the singers obtained by the acoustic instrumentation are both the coupling result of the vocal cords, the cavity, the vocal position and other factors. In fact, even though the third method can unmix vocal cord vibration with cavity resonance and other factors, its measurement method is intrusive and interfering, which already affects the vibration conditions and vibration state of the vocal cords. Furthermore, without the sound in the full range, the validity and scientificity of the measurement results are obviously limited.

According to patent data published by China National Intellectual Property Administration, Chinese patent applications relating to the measurement of vocal cord vibration include: CN200910021447.6 “detection system and detection method for mechanical properties of vocal cord vibration”; CN201210278461.6 “non-destructive measurement method of vocal cord vibration utilizing ultrasonic waveguide effect of natural sound channel”; CN201710673954.4 “method and system for classifying vibration patterns of vocal cords based on deep learning”; CN201310134285.3 “vocal cord detection device and signal detection method based on electrical impedance analysis”; CN201810762197.4 “method and system for analyzing vibration characteristics of vocal cords based on machine learning”; CN202111524676.7“method and system for obtaining image information for medical detection of vocal cords”; CN201210310811.2 “electroacoustic gating map measurement method of vocal cords and device using the same”; CN201220430883.6 “electroacoustic gating map measurement device of vocal cords”; CN91108961.6 “dynamic image analyzer for vocal cords”; CN201310676227.3 “ultrasonic laryngoscope system for electroacoustic gating chart synchronization and control method thereof”; CN201210036608.0 “bionic device for simulating vocal cord vibration”; and CN201110448314.4 “device for simulating vibration of human vocal cords and realization method thereof”, etc. The above patent applications either adopt a contact measurement method, a non-contact detection method, or a simulation method to detect the vibration characteristics of the vocal cords.

Chinese patent applications relating to resonance peaks are mainly focused on the measurement of speech resonance peaks, such as CN202210492452.0 “continuous speech resonance peak extraction method based on picking peaks”; CN202110273503.6 “determination method, device, electronic equipment and readable storage medium for speech resonance peaks”; CN201280076334.6 “resonance peak dependent speech signal enhancement”; CN202310882736.5 “speech recognition model training method, speech recognition method, device and apparatus”; CN201310087410.X “processing method and device for Doppler ultrasound pickup analysis”; CN200810224789.3 “objective evaluation method of singing tone purity based on audio spectral feature analysis”; CN202110063440.3 “speech resonance peak enhancement method based on deep learning”; CN201910687899.5 “extraction method for resonance peak feature of speech signal”; CN202010104431.X “resonance peak de-reverberation method in speech recognition”; CN202210658975.X “resonance peak feature extraction method for speech recognition”; and CN201810696459.8 “speech emotion recognition method based on resonance peak”. Most of the above patent applications use a microphone or other audio input device to collect speech signals, and analyze the resonance peak features of speech using Fourier transform and spectral analysis. Compared with the technical needs of voice type determination, the unmixing problem of cavity resonance and vocal cord vibration remains.

In summary, vocal cord vibration and singing voice type determination are important in singing acoustics, vocal physiology and clinical examination, but the conventional methods cannot truly reflect the vibration characteristics of the vocal cords in the natural singing state of the subject, and their accuracy, scientificity and non-destructiveness are obviously insufficient. Therefore, it is difficult to accurately determine the singing voice type.

In view of the inconvenience of measuring the vocal cords located within the human body, as well as the fact that the vocal cords need to be naturally relaxed for vibration, the laryngeal muscles should not be tensed, and there should be no external influence or intervention, there is an urgent need for a safer, more efficient, more scientific, and more accurate detection system and method for singing voice type determination.

### SUMMARY OF THE PRESENT INVENTION

An object of the present invention is to make up for the deficiencies of the conventional detection technology, and to provide a detection system and a method for singing voice type determination. The present invention can accurately determine the full-frequency domain parameters of steady vocal cord vibration, scientifically separate the vocal cord vibration from cavity resonance, and determine the singing voice type based on peak frequency band characteristics of the vocal cords.

Accordingly, in order to accomplish the above objects, the present invention provides:

A detection system for singing voice type determination, comprising: a vibration measurement system, a data acquisition system and a data management system.

The vibration measurement system comprises: a laser scanning head (wherein the laser scanning head comprises a built-in interferometer, a pair of pendulum mirrors for deflecting laser beams to a desired test position, and a color video camera for visualization; a laser emitted by the laser scanning head is a He-Ne laser with a wavelength of 632.8 nm), a morphology scanning unit (the morphology scanning unit send morphology data of a measured object into a vibration analysis software as a laser measurement point), and an optical measurement system (wherein the optical measurement system consists of an optoelectronic signal converter, an optical signal amplifier, and an optoelectronic signal processor, thereby converting a frequency signal into velocity and displacement signals of a vibrating object). It should be noted that the vibration measurement system adopts laser Doppler principle to measure changes of frequency, amplitude and other parameters during the vocal cord vibration, which has the advantages of high precision, fast dynamic response, large measurement range, non-contact measurement, anti-electromagnetic interference, and insensitivity to lateral vibration interference.

The data acquisition system is a front-end (with a built-in data acquisition and signal generator module), which demodulates a raw Doppler signal input from the laser scanning head through a velocity decoder (covering an entire dynamic frequency range from 0 to 100 kHz), and then sends a demodulated signal to a PC (the data management system) through a digital interface, so as to obtain a steady vibration signal of vocal cords containing velocity and displacement information.

The data management system consists of a synchronized acquisition control module, a data acquisition module, an imaging display module, a data storage module, a data processing module, and an acoustic parameter extraction module; the synchronized acquisition control module and the data acquisition module are configured to collect and record real-time data of vocal cord vibration during a singing process; the imaging display module provides real-time imaging and monitoring of the vocal cord vibration; the data storage module saves collected data to a computer hard disk; and the data processing module and the acoustic parameter extraction module process saved data with FFT (Fast Fourier Transform), as well as extract relevant vibration parameters in a resulting frequency domain diagram.

The laser scanning head is placed 1-3 meters right ahead a subject, preferably 2.5 meters. A top bandwidth limit for laser scanning is 8 kHz, and a maximum vibration speed is no less than 500 mm/s (peak value).

The present invention further provides a method for singing voice type determination, comprising steps of: first, obtaining a scale of an open accent “ah” from the subject; second, emitting a laser by the laser scanning head, aligning and illuminating the laser to external surfaces of vocal cords to be measured, and focusing; and then starting scanning measurement; third, inputting an optical signal of vocal cord vibration into a data acquisition system, and demodulating measurement laser Doppler frequency shift (f=2v/λ) information caused by the vocal cord vibration by a decoder; then converting a frequency signal into velocity and displacement signals of the vocal cord vibration by a signal processor; and then generating a real-time time-domain map and a spectrum map by a data management system; finally, determining a peak frequency range of the vocal cord vibration by analysis, and using the peak frequency range as a theoretical basis for the singing voice type determination.

Compared with the prior art, the present invention has the following beneficial effects:
(1) By measuring the steady vocal cord vibration during singing, the present invention avoids the conventional method in which the singing voice type is determined based on resonance peaks of singers, which means the vocal cord vibration is effectively unmixed with cavity resonance and other factors.
(2) The present invention adopts laser Doppler principle for real-time, non-invasive and efficient measurement of the vocal cord vibration during singing, which provides a novel scientific method for singing voice type determination by means of

In conclusion, the present invention adopts the advanced Doppler frequency shift signal technology and quantitative analysis, which maintains the natural vibration of the vocal cords, so as to obtain spectrograms of the vocal cord vibration in its full frequency band through contactless (non-destructive), accurate and efficient measurement.

### BRIEF DESCRIPTION OF THE DRAWINGS

FIG. 1 is a perspective view of a detection system for singing voice type determination;

FIG. 2 is a sketch view positioning a laser measurement point;

FIG. 3 is a spectrogram of vocal cord vibration of a bass singer;

FIG. 4 is a spectrogram of vocal cord vibration of a tenor singer;

FIG. 5 is a resonance peak spectrogram of the bass singer: and

FIG. 6 is a resonance peak spectrogram of the tenor singer.

### DETAILED DESCRIPTION OF THE PREFERRED EMBODIMENT

The present invention will be further descried hereinafter in connection with the accompanying drawings and embodiments.

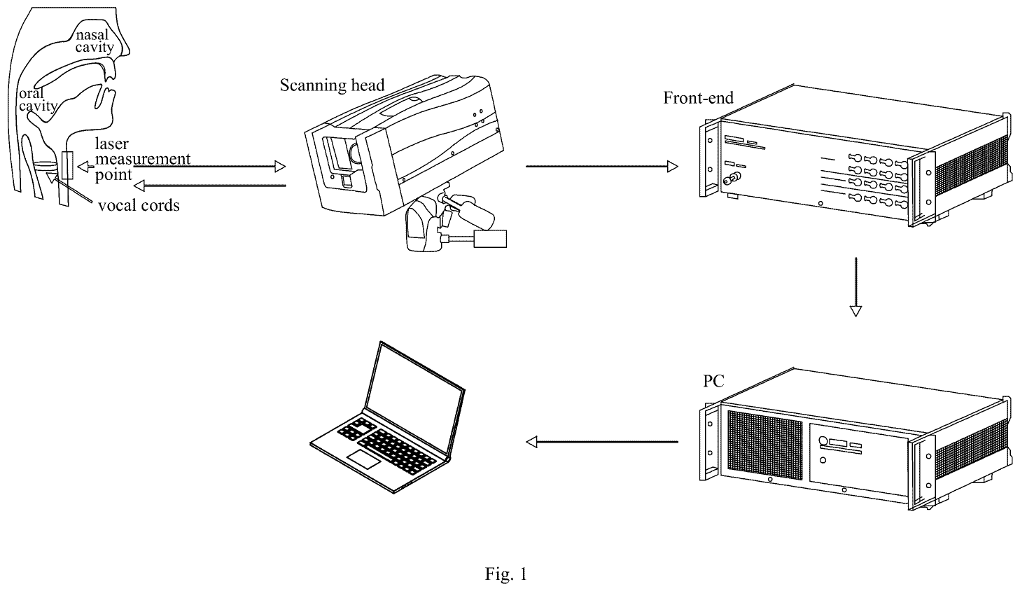

Referring to FIG. 1, a detection system for singing voice type determination is illustrated, comprising: a vibration measurement system, a data acquisition system and a data management system as mentioned above.

A related experiment comprises specific steps of:
(1) first, placing a laser scanning head 2.5 meters right ahead a subject; and obtaining a scale of an open accent “ah” from the subject;
(2) second, emitting a laser by the laser scanning head, aligning and illuminating the laser to external surfaces of vocal cords to be measured, and focusing; and then starting scanning measurement;
(3) third, inputting an optical signal of vocal cord vibration into a data acquisition system, and demodulating measurement laser Doppler frequency shift (f=2v/λ) information caused by the vocal cord vibration by a decoder; then converting a frequency signal into velocity and displacement signals of the vocal cord vibration by a signal processor;
(4) then, generating a real-time time-domain map and a spectrum map by a data management system; and
(5) finally, determining a peak frequency range of the vocal cord vibration by analysis, and using the peak frequency range as a theoretical basis for the singing voice type determination.

The detection system for singing voice type determination is suitable for all singers, whose principle and measurement method are universal.

### Embodiment 1

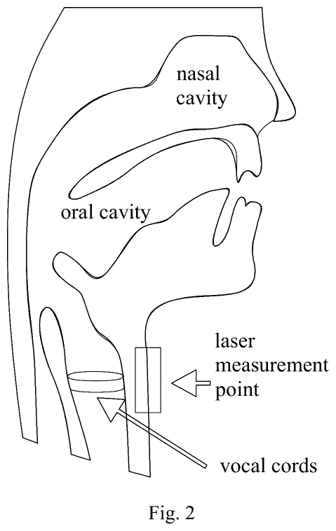

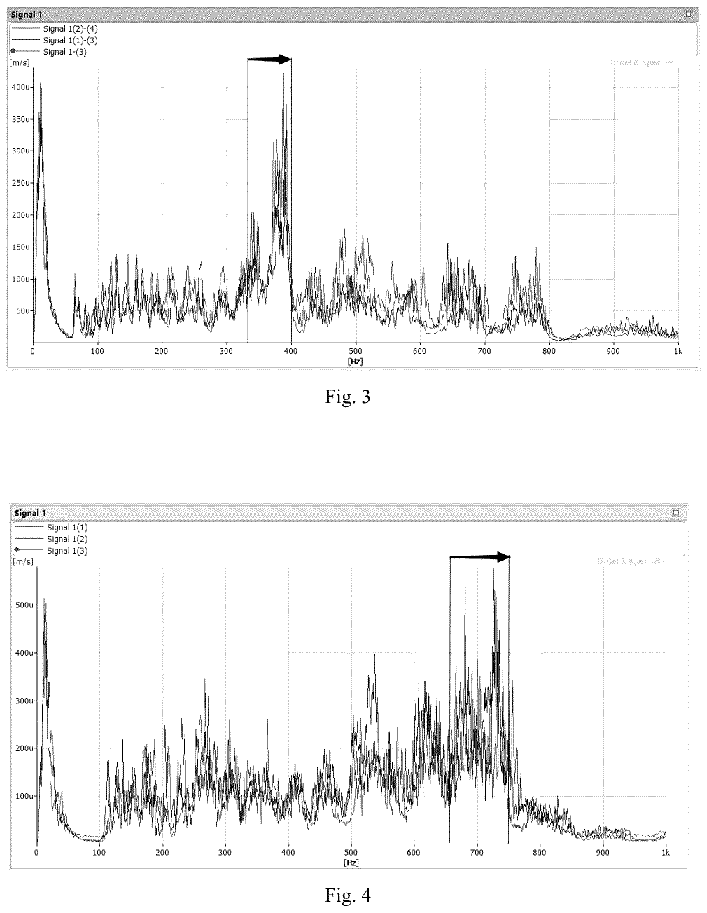

The present invention provides a detection system and a method for singing voice type determination. Herein, the present invention selects a general sample and uses the system as previously described, wherein:
(1) the tested vocal cords were selected from outstanding bass and tenor singers in the vocal performance program of a university; and
(2) the specific experimental steps were the same as in the previous specific embodiment; during optical measurement of vocal cord vibration, the external surface of the vocal cords of the subject was selected as the laser measurement point (as shown in FIG. 2), and after repeating the experiment three times, experimental results were obtained (as shown in FIGS. 3 and 4).

By observing the experimental spectrograms of the present invention (FIGS. 3 and 4), the bandwidth of the vibration peak frequency is selected for describing.

The frequency dynamic range of vocal cord vibration refers to the first peak to the last peak in the spectrogram, which represents the frequency range between the lowest and the highest pitch the person can reach, namely the tonal range. Although the resonance cavity of the human body can make the sound lower or higher and thus affects the subjective perception of the tonal range to a certain extent, it does not change the tonal range in physical sense (which depends on physiological structure and functional state of the vocal cords). Referring to FIGS. 3 and 4, it is visualized that during singing, the dynamic range of vocal cord vibration of the bass is 60 Hz-800 Hz, while that of the tenor is 110 Hz-950 Hz, which means the lowest frequency of vocal cord vibration of the bass, which is 60 Hz, is lower than that of the tenor, which is 110 Hz.

By observing the experimental spectrograms of the present invention (FIGS. 3 and 4), the frequency range where the energy is the strongest and most concentrated is selected for describing.

According to vibration principle, the largest amplitude indicates the strongest energy. Referring to FIGS. 3 and 4, it is visualized that the frequency range where the energy of the vocal cord vibration is strongest and most concentrated for the bass is about 330 Hz-400 Hz, and that for the tenor is about 660 Hz-750 Hz. Therefore, 330 Hz-400 Hz and 660 Hz-750 Hz are taken as the peak frequency bands of the vocal cord vibration of the bass and the tenor singers, respectively.

With a large number of samples, the present invention can subsequently provide test criteria for singing voice type determination, i.e., criteria for determining tenor, baritone, bass, soprano, mezzo-soprano, and contralto.

### Embodiment 2 (Comparison)

Furthermore, a comparison experiment was conducted in which the resonance peaks of the bass and tenor singers were measured using acoustic instruments.

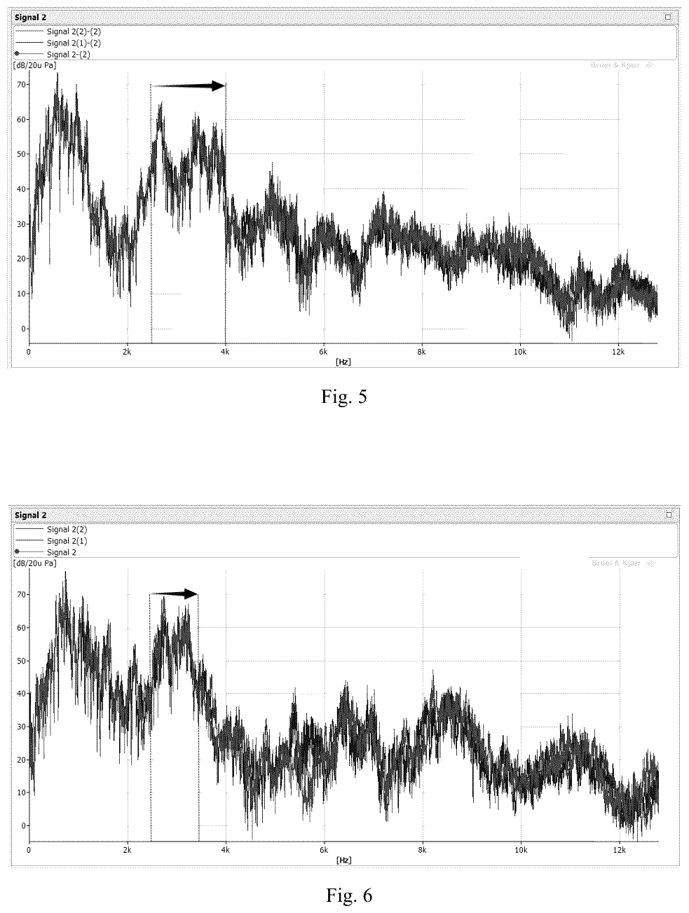

Comparison results are provided (as shown in FIGS. 5 and 6).

By observing the spectrograms of the bass and tenor singers (FIGS. 3 and 4), the resonance peaks thereof are selected for describing.

It is now widely recognized that the singer's resonance peak is a resonance wave peak in the frequency range of 2200-3200 Hz, which is mainly affected by the modulation of the resonance cavity in the case of steady vocal cord vibration, leading to energy redistribution (enhancement or attenuation) of certain frequencies. Referring to FIGS. 5 and 6, it is visualized that the resonance peaks of the bass singer range from 2500 Hz to 4000 Hz, and those of the tenor singer range from 2500 Hz to 3200 Hz.

Through the comparison, it is further verified that the resonance peak analysis of singers is unable to determine the singing voice type. There are three reasons: one is that such method cannot explain the large amplitude peak frequencies within 1000 Hz as shown in FIGS. 5 and 6; the second is that the spectrogram is the coupling result of the vocal cords and the cavities, and both of the vocal cords and the cavities are sound contributors and cannot be separated or unmixed; and the third is that it is impossible to find a pattern for the appearance of the resonance peak bands of each voice type.

It can be clearly observed from the measurement results of the embodiment 1 of the present invention that the peak frequency band in FIG. 3 is lower than that in FIG. 4, which is in accordance with the nature of bass and tenor. Therefore, the detection system and method of the present invention can be used for singing voice type determination.

What is claimed is:

1. A detection system for singing voice type determination, comprising: a vibration measurement system, a data acquisition system and a data management system;
wherein the vibration measurement system comprises: a laser scanning head, a morphology scanning unit, and an optical measurement system; wherein the laser scanning head comprises a built-in interferometer, a pair of pendulum mirrors for deflecting laser beams to a desired test position, and a color video camera for visualization; a laser emitted by the laser scanning head is a He-Ne laser with a wavelength of 632.8 nm; the morphology scanning unit send morphology data of a measured object into a vibration analysis software as a laser measurement point; wherein the optical measurement system consists of an optoelectronic signal converter, an optical signal amplifier, and an optoelectronic signal processor, thereby converting a frequency signal into velocity and displacement signals of a vibrating object;
wherein the data acquisition system is a front-end with a built-in data acquisition and signal generator module, which demodulates a raw Doppler signal input from the laser scanning head through a velocity decoder covering an entire dynamic frequency range from 0 to 100 kHz, and then sends a demodulated signal to the data management system through a digital interface, so as to obtain a steady vibration signal of vocal cords;
wherein the data management system consists of a synchronized acquisition control module, a data acquisition module, an imaging display module, a data storage module, a data processing module, and an acoustic parameter extraction module; the synchronized acquisition control module and the data acquisition module are configured to collect and record real-time data of vocal cord vibration during a singing process; the imaging display module provides real-time imaging and monitoring of the vocal cord vibration; the data storage module saves collected data to a computer hard disk; and the data processing module and the acoustic parameter extraction module process saved data with FFT (Fast Fourier Transform), as well as extract relevant vibration parameters in a resulting frequency domain diagram.

2. A method for singing voice type determination, comprising steps of:
(1) placing a laser scanning head 1-3 meters right ahead a subject; wherein a top bandwidth limit for laser scanning is 8 kHz, and a maximum vibration speed is no less than 500 mm/s;
(2) obtaining a scale of an open accent “ah” from the subject;
(3) emitting a laser by the laser scanning head, aligning and illuminating the laser to external surfaces of vocal cords to be measured, and focusing; and then starting scanning measurement;
(4) inputting an optical signal of vocal cord vibration into a data acquisition system, and demodulating measurement laser Doppler frequency shift information caused by the vocal cord vibration by a decoder; then converting a frequency signal into velocity and displacement signals of the vocal cord vibration by a signal processor; and
(5) generating a real-time time-domain map and a spectrum map by a data management system; then determining a peak frequency range of the vocal cord vibration by analysis, and using the peak frequency range as a theoretical basis for the singing voice type determination.

3. The method, as recited in claim 2, wherein in the step (1), the laser scanning head is placed 2.5 meters right ahead the subject.
